(19) United States
(12) Patent Application Publication
(10) Pub. No.: US 2017/0080256 A1
(43) Pub. Date: Mar. 23, 2017

(54) BRAIN TO BRAIN INTERFACE SYSTEM APPLIED TO SINGLE BRAIN

(71) Applicant: KOREA INSTITUTE OF SCIENCE AND TECHNOLOGY, Seoul (KR)

(72) Inventors: Hyung Min KIM, Seoul (KR); Seung-Jong KIM, Seoul (KR); Inchan Youn, Seoul (KR); Jong Min LEE, Seoul (KR)

(73) Assignee: KOREA INSTITUTE OF SCIENCE AND TECHNOLOGY, Seoul (KR)

(21) Appl. No.: 15/262,370

(22) Filed: Sep. 12, 2016

(30) Foreign Application Priority Data
Sep. 17, 2015 (KR) 10-2015-0131508

(51) Int. Cl. A61N 7/00; A61B 5/00; A61B 5/0476

(52) U.S. Cl. CPC A61N 7/00; A61B 5/0476; A61B 5/4836; A61N 2007/0026

(57) ABSTRACT

A brain to brain interface system has a brain activity detection device configured to detect activity state information of a brain, a brain stimulation device configured to stimulate an area of at least a part of the brain to activate or inactivate brain cells of the corresponding area, and a computer configured to control the brain activity detection device and the brain stimulation device, wherein brain activity state information of a subject's brain (“a target brain”) is obtained through the brain activity detection device, and an area of at least a part of the target brain is stimulated through the brain stimulation device based on the brain activity state information of the target brain to regulate a function of the target brain.

### CROSS-REFERENCE TO RELATED APPLICATION

This application claims priority to Korean Patent Application No. 10-2015-0131508, filed on Sep. 17, 2015, and all the benefits accruing therefrom under 35 U.S.C. §119, the contents of which in its entirety are herein incorporated by reference.

### BACKGROUND

1. Field

The present disclosure relates to a brain interface system for regulating brain function, and more particularly, a brain to brain interface system between single brains that measures brain activity state information, and applies stimuli to a corresponding brain based on the measured brain activity state information to regulate brain function.

[Description about National Research and Development Support]

This study was supported by the Korea Health Technology R&D Project through the Korea Health Industry Development Institute (KHIDI), funded by the Ministry of Health & Welfare of the Republic of Korea (grant HR14C0007). This research was also supported by the Brain Research Program through the National Research Foundation of Korea (NRF) funded by the Ministry of Science, ICT & Future Planning (grant 2015M3C7A1064833).

2. Description of the Related Art

The brain is responsible for physical, sensory, and all other metal activities of living things.

The brain has functional areas for different functions in each part, and a tract for signal transmission is formed between the functional areas to transmit brain neural signals. This signal transmission pathway is termed a “brain neural circuit”.

FIG. 1 shows certain functional areas of the brain.

A typical functional area of the brain 1 includes a motor neural circuit related to movements of living things and a sensory neural circuit related to senses.

As shown in FIG. 1, the motor neural circuit is formed such that a prefrontal association area 21 responsible for high-level mental functions such as judgment and prediction, a premotor and supplementary motor area 22 involved in planning movements and responsible for unconscious movements or tension, and a primary motor area 23 involved in propagating motor commands to motor nerves throughout the body are connected through a neural signal transmission tract 26 to transmit information (neural signals).

The sensory neural circuit leads to a sensory area 25, a multisensory area 24 and a prefrontal association area 21 such as a somatosensory association area, an auditory association area, and a visual association area.

In addition, the brain 1 has a plurality of unexplained brain neural circuits related to emotional or mental activities.

These brain neural circuits act in combination to allow living things to do normal life activities.

If necrosis occurs at a part of the brain, causing a stroke, function disorders may occur, such as paralysis of the part of the body. Even though a mechanical injury or damage such as brain necrosis does not occur, if a specific brain neural circuit fails to operate normally, psychiatric diseases may occur, for example, persistent auditory or visual hallucinations.

In addition to these diseases caused by functional disorders of the brain, mild or severe physical symptoms caused by brain malfunction appear.

Drug treatment is being widely used to treat the diseases, but it is difficult to expect a prompt and direct symptom alleviation effect, and persistence may reduce due to limited drug dosing or adverse effects.

To provide a direct effect on the alleviation of a specific symptom, studies have been made on methods for applying mechanical stimuli directly to the brain.

However, a brain stimulation system according to related art applies stimuli to a predefined location with an aim of alleviating a predefined symptom, so its range of applications is very limitative. Further, stimuli are unilaterally applied without considering a situation in which the brain operates, which may rather cause damage to the brain.

### SUMMARY

The present disclosure is designed to solve the foresaid problem, and therefore, the present disclosure is directed to providing a system that delivers tailored stimuli suited to a situation in which the brain operates, and delivers prompt and optimized stimuli to regulate brain function.

To achieve the object, according to an aspect of the present disclosure, there is provided a brain to brain interface system including a brain activity detection device configured to detect activity state information of a brain, a brain stimulation device configured to stimulate an area of at least a part of the brain to activate or inactivate brain cells of the corresponding area, and a computer configured to control the brain activity detection device and the brain stimulation device, wherein brain activity state information of a subject's brain (“a target brain”) is obtained through the brain activity detection device, and an area of at least a part of the target brain is stimulated through the brain stimulation device based on the brain activity state information of the target brain to regulate a function of the target brain.

According to an embodiment, the computer may identify activity state of a brain neural circuit for performing a specific function through the brain activity state information, and allow the brain stimulation device to stimulate an abnormally activated area or an inactivated area in the brain neural circuit to inactivate or activate the corresponding area.

According to an embodiment, the computer may identify activity state of a brain area for performing a specific function through the brain activity state information, and allow the brain stimulation device to stimulate other brain area for performing a different function from the specific function to activate or inactivate the other brain area.

According to an embodiment, an affected part of nervous necrosis may be present in the target brain, and the brain stimulation device may stimulate an arbitrary part of the target brain to remedy an unbalanced state of the entire target brain caused by the presence of the affected part.

According to an embodiment, the brain stimulation device may simultaneously stimulate an affected side in which the affected part is present and an unaffected side in which the affected part is absent, such that the affected side and the unaffected side are in opposite activity state.

According to an embodiment, an affected part of nervous necrosis may be present in the target brain, in which a signal transmission tract of a brain neural circuit for performing a specific function is blocked by the affected part, and when activity state information of a high-level function area involved in giving a command for the specific function is obtained, the computer may allow the brain stimulation device to stimulate an arbitrary of the target brain, to help form a new signal transmission tract to bypass the affected part.

According to an embodiment, the brain stimulation device may simultaneously stimulate a low-level function area involved in executing the command in the brain neural circuit, and a surrounding area of the affected part.

According to an embodiment, an affected part of nervous necrosis may be present in the target brain, in which a signal transmission tract of a brain neural circuit for performing a specific function is blocked by the affected part, and when activity state information of a high-level function area involved in giving a command for the specific function is obtained, the computer may allow the brain stimulation device to stimulate a low-level function area involved in executing the command in the brain neural circuit to help perform the function.

According to an embodiment, the brain stimulation device may be a low intensity focused ultrasound device, which brings low intensity ultrasound beams into convergence to at least one focus.

According to an embodiment, the low intensity focused ultrasound device may move a position of the focus three-dimensionally.

### BRIEF DESCRIPTION OF THE DRAWINGS

FIG. 1 shows certain functional areas of the brain.

FIG. 2 is a conceptual diagram of a brain to brain interface system according to an embodiment of the present disclosure.

FIGS. 3 and 4 are diagrams illustrating a configuration of a low intensity focused ultrasound stimulation device according to an embodiment of the present disclosure.

FIG. 5 is a diagram illustrating piezoelectric effect of a piezoelectric element used in an ultrasound stimulation device.

FIGS. 6 and 7 show an ultrasound beam focused by a low intensity focused ultrasound stimulation device according to an embodiment of the present disclosure.

FIGS. 8 and 9 show adjustment of the focus position of low intensity ultrasound beams in a low intensity focused ultrasound stimulation device according to an embodiment of the present disclosure.

FIG. 10 shows an example of applying stimuli to a target brain using the brain to brain interface system of FIG. 2.

FIGS. 11 through 13 each show an example of application of the brain to brain interface system of FIG. 2 in the case where an affected part of nervous necrosis is present in a target brain.

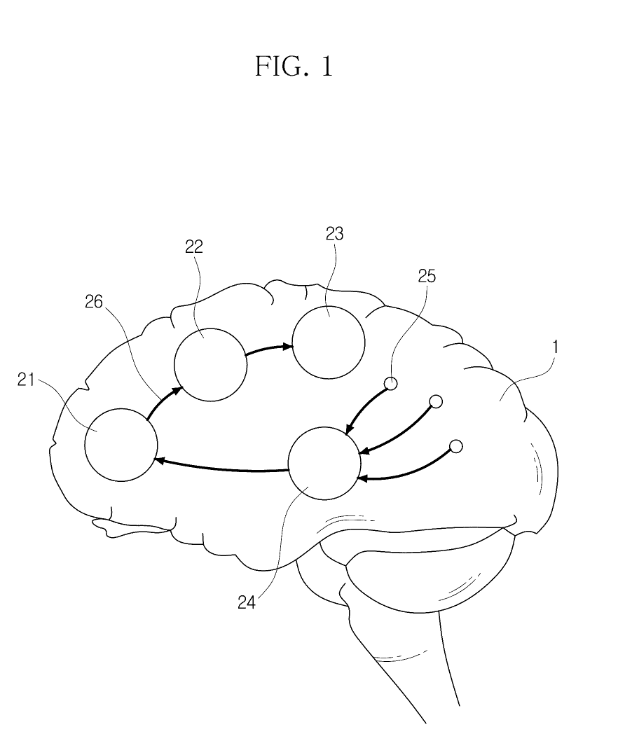

### DETAILED DESCRIPTION

Exemplary embodiments of the present disclosure will be hereinafter described with reference to the accompanying drawings. Although the present disclosure is described with reference to the embodiments shown in the drawings, it is described as an example, and the technical spirit of the present disclosure and key elements and their operation is not limited thereby.

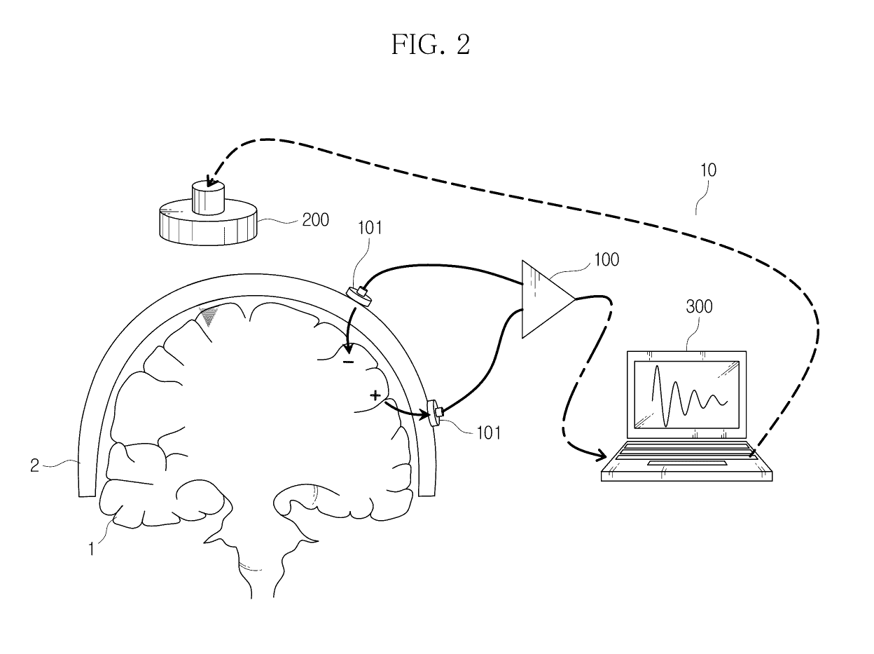

FIG. 2 is a conceptual diagram of a brain to brain interface system (hereinafter, abbreviated to a “system”) 10 according to an embodiment of the present disclosure.

As shown in FIG. 2, the system 10 includes a brain activity detection device 100 which detects activity state information of a subject's brain (“a target brain”) 1, a brain stimulation device 200 which stimulates an area of at least a part of the target brain 1 to activate or inactivate brain cells of the corresponding area, and a computer 300 which controls the brain activity detection device 100 and the brain stimulation device 200.

The system 10 according to this embodiment obtains brain activity state information the target brain 1 through the brain activity detection device 100, and immediately stimulates an area of at least a part of the target brain 1 through the brain stimulation device 200 based on the brain activity state information of the target brain 1 to regulate functions of the target brain 1.

The brain activity detection device 100 according to this embodiment may be an electroencephalogram (EEG) detection device, which non-invasively measures brainwaves through a plurality of electrodes 101 attached to a head surface 2. The plurality of electrodes 101 is attached to a location selected as being where activation takes place well in the brain according to MCN standard electrode position nomenclature.

Brainwaves are obtained by measuring signals from the brain surface on which electrical signals generated from numerous brain cells are manifested after being synthesized. The brainwave signals change spatially and temporally depending on brain activity and brain function. The brainwave signals have spatial properties related to brain function.

After measuring brainwaves, to obtain information using them, an operation for analyzing the brainwaves is needed.

Brainwaves are complex signals as a combination of signals in many frequency bands. Brainwaves are classified into delta, theta, alpha, beta, and gamma waves according to the ranges of frequencies and voltages. Delta (δ) waves have frequency of 0.1-3 Hz and amplitude of 20-200 μV, theta (θ) waves have frequency of 4-7 Hz and amplitude of 20-100 μV, and alpha (α) waves have frequency of 8-12 Hz and amplitude of 20-60 μV. Beta (β) waves have frequency of 12-30 Hz and amplitude of 2-20 μV, and gamma (γ) waves have frequency of 30-50 Hz and amplitude of 2-20 μV.

For brain activity state analysis using brainwaves, waves in a specific frequency band are primarily used, but it is necessary to set a desired frequency band because frequency characteristics of brainwaves differ in each person.

In brainwaves, an event-related potential (ERP) refers to an electrical activity of the brain that occurs during a predetermined period of time in response to a stimulus of specific information. Through many studies, among components of ERP, P300 has been reported as being related to many various cognitive activities such as decision-making, probability of signals, attention, discrimination, resolution of uncertainty, relevance of stimuli, and transmission of information.

To analyze brainwave characteristics in specific state, a power spectrum distribution describing the overall distribution of power for each frequency component is first observed, and brain activity state is determined through changes of components. The power spectrum distribution shows different aspects in each site of measurement on the head surface. For example, the occipital lobe corresponding to the back of the head has the primary visual cortex and is responsible for primary visual information, and the parietal lobe corresponding to the proximity of the crown of the head has the somatosensory cortex and is responsible for motor/sensory related information processing.

Through power spectrum analysis, increases and decreases of intensity in a specific frequency section can be seen. For example, through analysis of signals of a related brain area during arm movements, it is known that a phenomenon appears in which the signal intensity increases in 0.5-8 Hz band (ERS, event-related synchronization), and the signal intensity decreases in 9-22 Hz (ERD, event-related desynchronization).

Using this feature, it is possible to analyze how the intensity in a specific frequency band changes depending on the location in the brain, how signals are transmitted and received between each area of the brain or which areas are related to each other (That is, it is possible to analyze activity states of brain neural circuits).

Through many experiments of the brain, activity state information of various brain neural circuits and areas may be collected, and a brainwave model (reference brain activity state information) constructed by taking an average of the collected information may be stored in the computer 300.

The brainwave information measured by the EEG detection device is pre-processed through a filter and an amplifier (not shown), converted to digital signal through a converter (not shown), and inputted to the computer 300.

The computer 300 identifies the activity state of a specific brain neural circuit and/or a specific area of the target brain by comparing the brainwave data measured by the EEG detection device to the brainwave model through a conversion algorithm. Because frequency characteristics of brainwaves differ in each person, the brainwave data of the target brain may be optimally scaled and applied to the brainwave model.

According to this embodiment, although the EEG detection device is used as the brain activity detection device 100, the present disclosure is not limited thereto.

When a specific part of the brain is activated, oxygen is consumed, so oxygen is delivered through oxyhemoglobin, and again, an amount of oxygen rather increases higher than before, and as a device for detecting brain activity by measuring this change, a functional magnetic resonance imaging (fMRI) device and a near infrared spectroscopy (NIRS) device may be used.

Also, as an electric current flows, a magnetic field changes, and using the nature of the magnetic field that induces a current in a coil, a magnetoencephalography (MEG) device may be used to measure brain activity. Also, functional transcranial Doppler sonography (fTCD) may be used to detect brain activity by measuring a blood flow rate changing depending on brain activity state using the Doppler effect.

Many studies have been made on a system named “brain computer interface (BCI)”, in which activities of brain are directly inputted to a computer by a direct connection between the brain and the computer, allowing communication with the computer. Through BCI technology, activity state and activity intent of the target brain can be determined.

The computer 300 identifies the activity state of the specific brain neural circuit and/or the specific area of the target brain, and based on this, calculates a control signal for controlling the brain stimulation device 200 and controls the brain stimulation device 200.

According to this embodiment, as the brain stimulation device 200, a low intensity focused ultrasound device (herein referred to as an “ultrasound stimulation device”) is used.

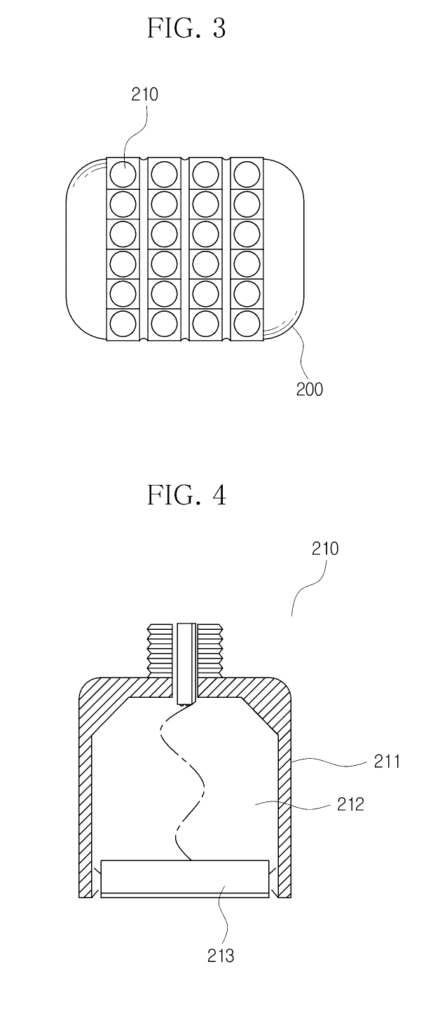

FIGS. 3 and 4 are diagrams illustrating a configuration of the ultrasound stimulation device 200 according to an embodiment of the present disclosure.

The ultrasound stimulation device 200 is configured such that a plurality of transducers 210 is arranged in array form.

A function module (not shown) including a signal generator to generate voltage signals which are applied to the transducers 210 and an amplifier to amplify signals is connected to the ultrasound stimulation device 200, and the function module is connected to the computer 300.

Although the ultrasound stimulation device 200 according to this embodiment has the plurality of transducers 210 arranged in matrix form, various modifications may be made, for example, the transducers 210 arranged in circular ring shape.

Each piezoelectric element 213 of each transducer 210 outputs ultrasound having spatial peak pulse average intensity (Isppa) less than spatial peak temporal average intensity (Ispta) of 3 W/cm² that does not do harm to the body. The low intensity ultrasound overlaps, creating low intensity ultrasound beams.

A plurality of ultrasound stimulation devices 200, 200′ is attached to the inside of a helmet shaped headwear, and when the subject wears the headwear on the head, the plurality of ultrasound stimulation devices 200, 200′ may be fixed toward the head of the subject.

The transducers 210 are all arranged in the front direction, and to bring the low intensity ultrasound beams to one focus F, a phase shift is introduced between spherical ultrasound waves generated by the piezoelectric elements 213. Its detailed description will be provided later.

FIG. 4 is a diagram illustrating a configuration of the transducer 210 according to this embodiment.

As shown in FIG. 4, the transducer 210 according to this embodiment includes a body 211, which is open on one side and the piezoelectric element 213 formed in the opening of the body 211. An inner part 212 of the body 211 is filled with air. Each piezoelectric element 213 is connected to a wire to apply voltage to the piezoelectric element 213. The body 211 is formed with a size for fixing one piezoelectric element 213.

According to this embodiment, the piezoelectric element 213 uses a material that generates piezoelectric effect such as quartz and turmaline, and the transducer 210 produces and outputs ultrasound using the piezoelectric effect of the piezoelectric element 213.

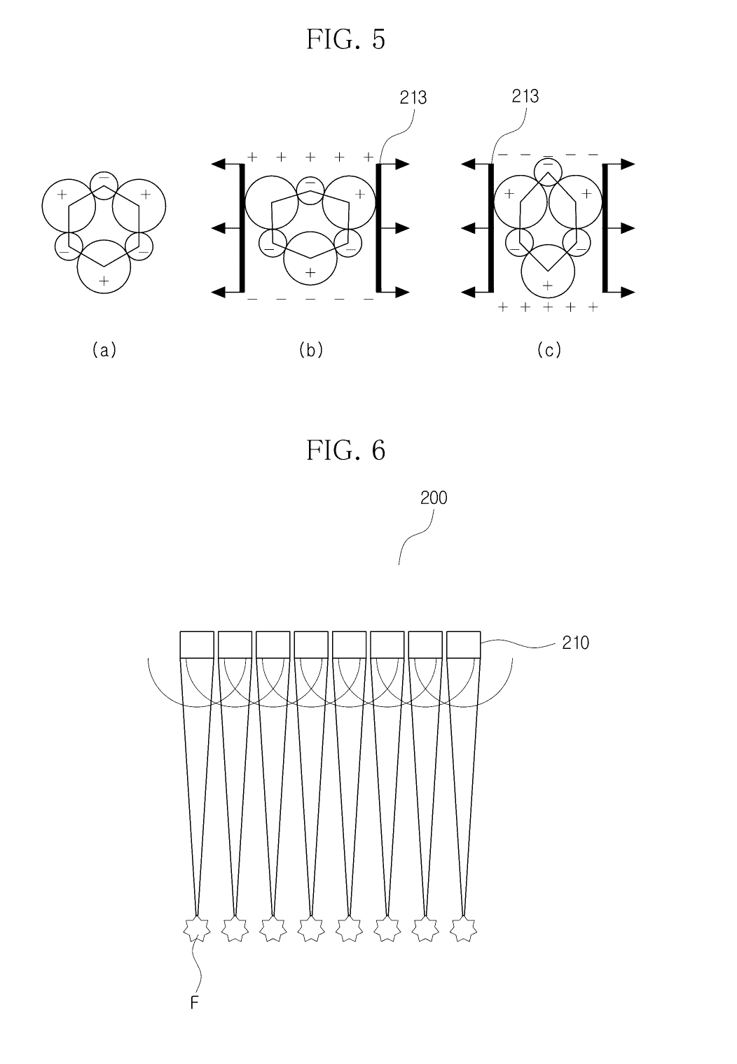

FIG. 5 is a diagram illustrating the piezoelectric effect of the piezoelectric element 213.

As shown in FIG. 5, when tension and compression is repeatedly applied along one axis of the piezoelectric element 213 of quartz crystals, positive charges (+) are generated on one side and negative charges (−) are generated on the other side, producing an electric current.

This polarization phenomenon at the piezoelectric element 213 takes place when the crystal structure becomes distorted and a shift in relative position between (+) ions and (−) ions occurs. The center of gravity of charges having undergone position shifting within the element is automatically corrected, but an electric field is created between two faces of a crystal. The direction of the electric field is opposite under compression and tension.

On the contrary, when voltage is applied to two faces of the piezoelectric element 213, (+) ions in the electric field move to (−) electrode, and (−) ions move to (+) electrode. By this converse piezoelectric effect, the piezoelectric element 213 is induced to stretch and contract depending on the direction of voltage applied from the exterior.

As elongation and contraction of the piezoelectric element 213 repeats, ultrasound having frequency above the audible range is produced by a similar principle to the speaker's principle of operation.

As best shown in FIG. 3, the ultrasound stimulation device 200 according to this embodiment is a phased array device in which the plurality of transducers 210 is arranged and each transducer independently receives applied voltage signals and outputs ultrasound.

According to this embodiment, the low intensity ultrasound beams outputted from the piezoelectric elements 213 of each transducer 210 converge to at least one focus F using an overlap phenomenon of ultrasound.

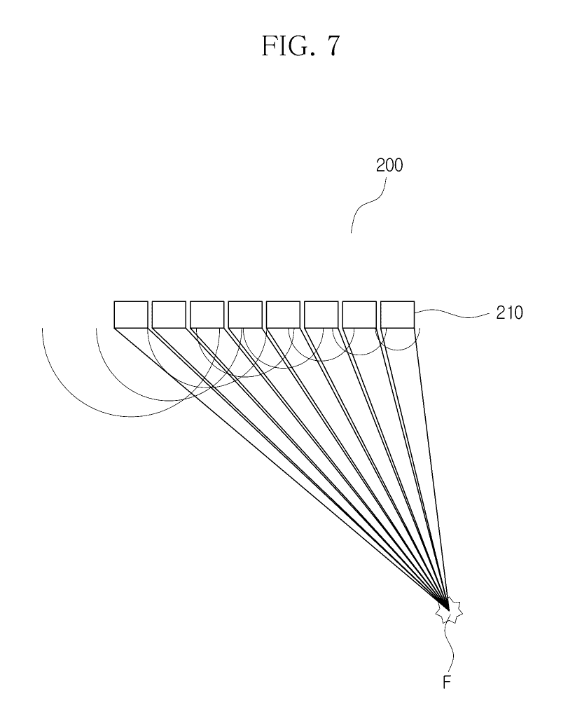

FIGS. 6 and 7 show the focused ultrasound beam.

As shown in FIG. 6, each transducer 210 generates spherical ultrasound waves, and an overlap occurs between spherical ultrasound waves generated by the transducers 210.

This overlap phenomenon forms low intensity ultrasound beams converging to focus F located a predetermined distance from the ultrasound stimulation device 200.

FIG. 6 is the case in which a phase shift is not introduced between spherical waves generated by the transducers 210, and ultrasound beams are vertically sent from each transducer 210 toward each focus F.

In contrast, as shown in FIG. 7, when a phase shift is introduced between spherical ultrasound waves generated by the transducers 210, low intensity ultrasound beams are brought into convergence to one focus F.

Also, when the phase shift between spherical ultrasound waves generated by the transducers 210 is regulated, the position of the focus F may be adjusted.

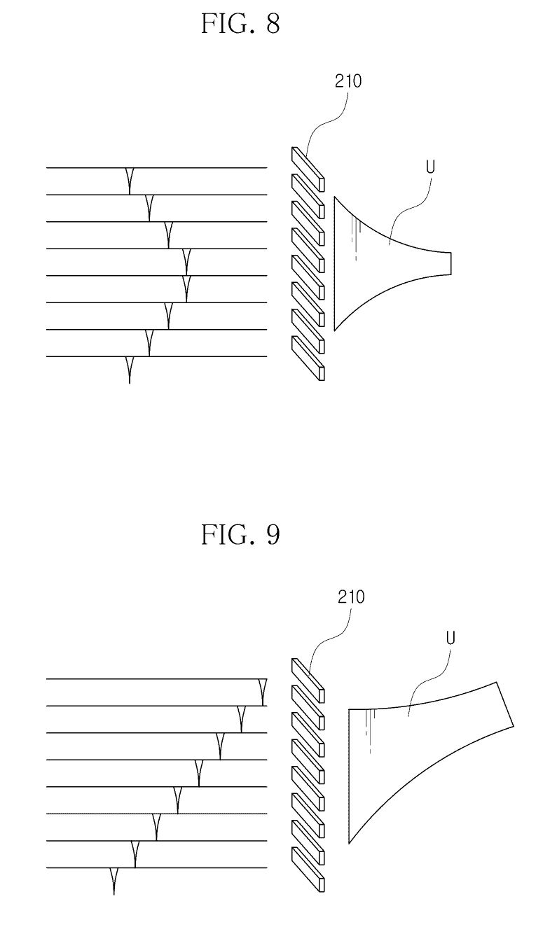

FIGS. 8 and 9 show adjustment of the position of the focus F.

The graphs shown on the left side of FIGS. 8 and 9 show voltage signals applied to each transducer 210 at a time interval.

As shown in FIGS. 8 and 9 for comparison, when the time interval between voltage signals applied to each transducer 210 changes, the phase shift between spherical ultrasound waves generated by the transducers 210 changes and the position of the focus F changes. The position of the focus F may be adjusted three-dimensionally in anterior-posterior, horizontal, and vertical directions.

Stimulation through low intensity focused ultrasound stimulates a site located at the focus of ultrasound beams.

The computer 300 sets coordinates of a target area (focus position) to be stimulated by the brain stimulation device 100 to allow the brain stimulation device 100 to accurately stimulate the corresponding area.

The coordinates of the target area may be set based on a known brain map, and may be set based on a brain map unique to the target brain constructed through precise examination of the target brain.

A device, which stimulates brain cells through the magnetic field or electric current, may be used as the brain stimulation device, while the use of an ultrasound stimulation device capable of focusing low intensity ultrasound can accurately focus on a specific brain area to selectively deliver local stimulation to the corresponding area.

Through ultrasound stimulation, if pulsed electrical signals applied as input to an ultrasound stimulator are modulated, it is possible to stimulate an area of at least a part of the brain to activate or inactivate brain cells of the corresponding area.

It is known that the low intensity focused ultrasound stimulation according to an embodiment of the present disclosure purely transmits mechanical energy to cells without heat generation, and acts on ion channels involved in neurotransmission or gets involved in neuromodulation through changes in cell membrane capacitance.

According to the system 10 constructed as above, tailored stimuli suited to brain activity state of the target brain can be applied in various forms.

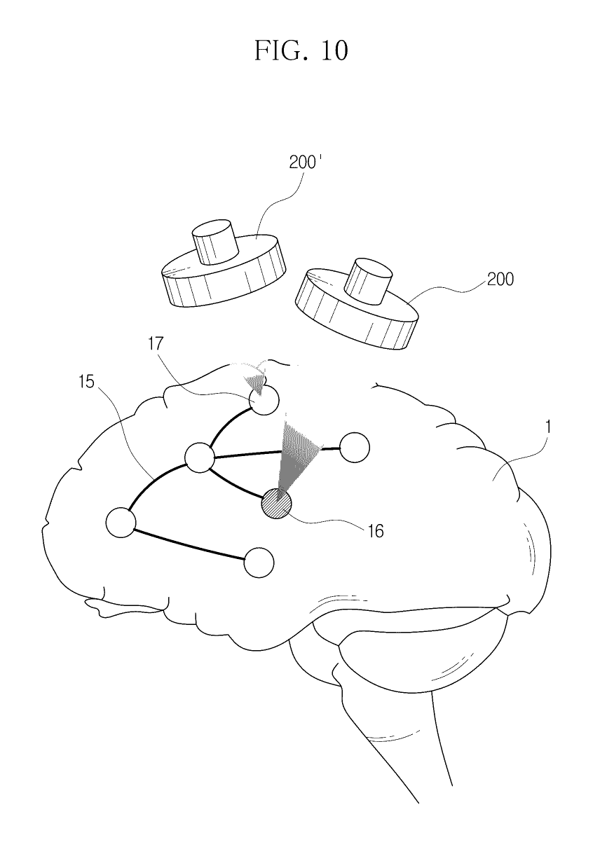

FIG. 10 shows an example of applying stimuli to the target brain 1 using the system 10.

As shown in FIG. 10, the computer 300 identifies the activity state of a brain neural circuit 15 for performing a specific function through the brain activity state information of the target brain 1.

The computer 300 compares the activity state information of the brain neural circuit 15 to the reference brain activity state information, and determines whether the brain neural circuit 15 normally operates. If an abnormally activated area 17 or an inactivated area 16 is present in the brain neural circuit 15, the corresponding area is stimulated through the brain stimulation device 100 to inactivate or activate the corresponding area.

For example, the brain neural circuit 15 may be a brain neural circuit related to a digestive function. In the case of patients suffering from indigestion due to poor movements of digestive organs and secretion of digestive juices in excessively large amounts, an area involved in movement of digestive organs is activated by stimulation, and an area involved in promoting digestive juice secretion is inactivated by stimulation, to alleviate indigestion. As shown in FIG. 10, stimulation to the activated area 17 and the inactivated area 16 can be accomplished through the plurality of brain stimulation devices 200, 200′ at the same time.

Upon stimulation of the brain stimulation device 200, information associated with the activated or inactivated state of the site of stimulation is fed back through the brain activity state information detected through the brain activity detection device 100, to allow the brain stimulation device 200 to adjust the stimulation intensity and location in real time.

On the other hand, the computer 300 may identify the activity state of the brain neural circuit 15 for performing a specific function through the brain activity state information, and apply stimuli to activate or inactivate a brain neural circuit for performing a different function from the corresponding specific function.

For example, the brain neural circuit 15 may be a neural circuit for perceiving an object while seeing the object. If the subject is a patient who feels extremely fearful when seeing a specific object, it is said that neural circuits for fear other than the brain neural circuit 15 abnormally operate.

The computer 15 detects the activity of the neural circuits for fear other than the brain neural circuit 15 at the target brain 1, and induces the inactivation of the neural circuits for fear through the brain stimulation device 200, to alleviate the corresponding symptom.

Although the above disclosure describes that the system 10 according to this embodiment is used to alleviate mental or physical diseases or symptoms, the present disclosure is not limited thereto.

For example, when the subject sees a photo of a snow covered view, it is possible to stimulate a sensory neural circuit through the brain stimulation device 200 to allow the subject to have a feeling of cool sensation.

That is, the system 10 according to this embodiment creates virtual experiences by disturbing some of brain functions based on real brain activity state information of the target brain, and can be variously used in the entertainment field or a variety of fields.

As the system 10 according to this embodiment can accurately stimulate a desired location of the target brain 1 using focused ultrasound, it can be usefully used in assisting normal activities of the target brain 1 in case that an affected part 20 of nervous necrosis caused by a stroke occurs in the target brain 1.

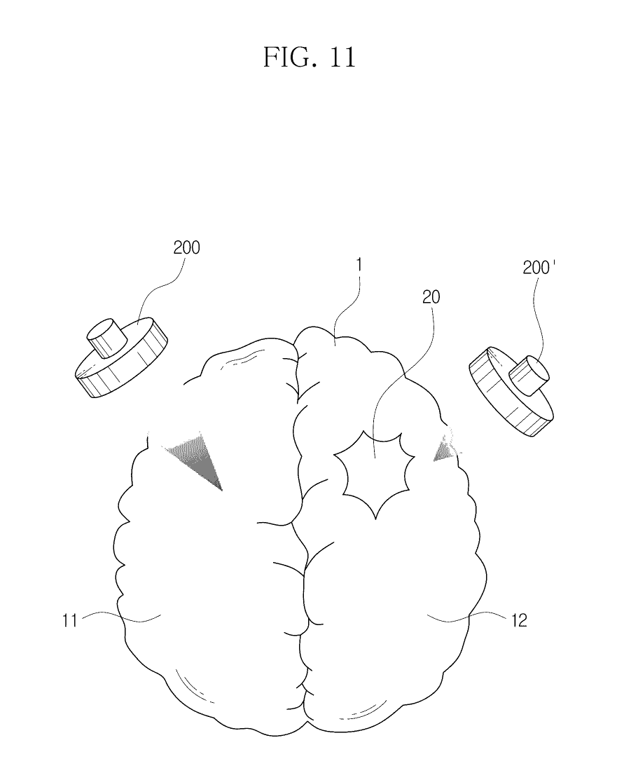

FIGS. 11 through 13 show an example of application of the system 10 when the affected part 20 of nervous necrosis is present in the target brain 1.

The brain tends to operate into balance of overall activity state. For example, during movements of a right hand, brainwave activity of a left hemisphere is suppressed, while activity of a right hemisphere is activated, and during movements of a left hand, a contrary phenomenon occurs.

When the affected part 20 of nervous necrosis occurs in the target brain 1, the target brain 1 is placed in an unbalanced state due to the presence of the affected part 20, and because of this unbalance, a variety of adverse effects may occur.

According to this embodiment, the brain stimulation devices 200, 200′ stimulate an arbitrary part of the target brain 1 to correct the overall unbalanced state of the target brain 1 caused by the presence of the affected part 20.

Referring to FIG. 11, this is the case where the affected part 20 occurred at the right hemisphere (the affected side) 12 of the target brain 1, and the affected part did not occur at the left hemisphere (the unaffected side) 11.

The system 10 according to this embodiment stimulates the affected side 12 and the unaffected side 11 simultaneously using the plurality of brain stimulation devices 200, 200′ such that the activity state of the two sides are opposite, to mitigate the overall unbalanced state of the target brain 1.

For example, when brain damage is not severe, stimuli are applied to activate the unaffected side 11 while inactivating the affected side 12, and when brain damage is severe, stimuli are applied to inactivate the unaffected side 11 while activating the affected side 12.

By applying a brain stimulation protocol properly depending on the brain state, the effect on the rehabilitation or treatment of brain damage can be enhanced.

Although FIG. 11 shows that the two brain stimulation devices 200, 200′ stimulate an arbitrary part of the target brain 1 using focused ultrasound, the present disclosure is not limited thereto.

More than two brain stimulation devices may be used, and the brain stimulation device may exert the influence of ultrasound on the whole target brain 1 without ultrasound focusing.

In focusing ultrasound, the focal position may be located on an arbitrary area of the target brain 1 that is not set, and may be located on an area at an optimized location derived to be effective in maintaining balance in activity of the target brain 1 though repeated experimentation.

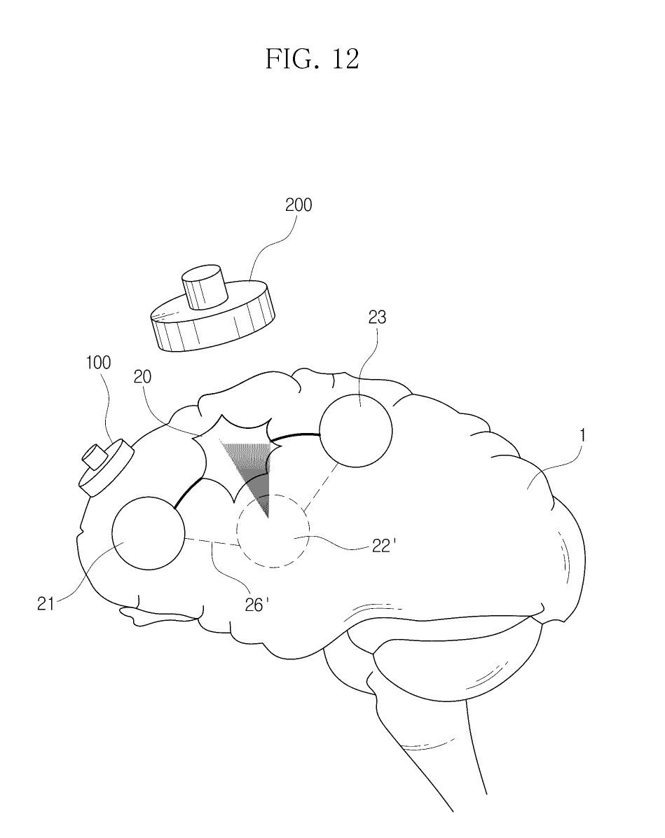

FIG. 12 is a diagram illustrating another example of application of the system 10 to the target brain 1 in which the affected part 20 of nervous necrosis is present.

A signal transmission tract of a brain neural circuit for performing a specific function may be blocked by the affected part 20 occurred in the target brain 1.

For example, the brain neural circuit for performing a specific function is a motor neural circuit.

As shown in FIG. 12, the signal transmission tract running from the prefrontal association area 21 to the primary motor area 23 is blocked by the affected part 20. The affected part 20 occurs at the premotor and supplementary motor area 22, and the function of the premotor and supplementary motor area 22 is lost.

As the tract of neural signal transmission is blocked by the affected part, conscious motor signals intended by the prefrontal association area 21 are not transmitted.

Similar to other organs, the brain experiences reconfiguration of neural circuits when continuously subjected to stimuli. It is termed “brain plasticity”.

Currently, stimuli are applied to the brain through physical and/or drug treatment to promote brain plasticity, helping rehabilitation of patients suffering from stoke. However, this method has a slow effect and may cause other adverse effects.

According to this embodiment, the computer 300 determines whether command signals for operating a brain neural circuit for performing a specific function are generated at a high-level function area of the corresponding brain neural circuit, by analyzing the brain activity state information obtained through the brain activity detection device 100.

For example, the prefrontal association area 21 is a high-level function area from which motor commands of organs such as hands are generated in the motor neural circuit.

The computer 300 determines whether there is an intent to move the body, by analyzing brain activity state information of the prefrontal association area 21.

If it is in normal state, command signals generated from the prefrontal association area 21 are transmitted to the primary motor area 23 through the motor signal circuit, causing movements. However, the corresponding signal transmission is blocked by the affected part 20.

When an intent to make physical movements is detected in the prefrontal association area 21, the computer 300 immediately stimulates an arbitrary part of the target brain 1 using the brain stimulation device 200, to help form a new signal transmission tract 26′ to bypass brain signals generated from the prefrontal association area 21 to the primary motor area 23 (promoting reconfiguration of neural circuits).

According to this embodiment, when a motor intent is generated from the prefrontal association area 21, in response, stimuli are applied to an arbitrary part of the target brain 1, and thus, the target brain 1 easily recognizes the site of stimulation as an area related to the prefrontal association area 21, leading to promotion of brain plasticity.

If stimuli are continuously applied to the same site, the corresponding site may be reconfigured to function as a new premotor and supplementary motor area 22′ in place of the damaged premotor and supplementary motor area 22 by brain plasticity, inducing complete recovery of the function lost by the affected part 20.

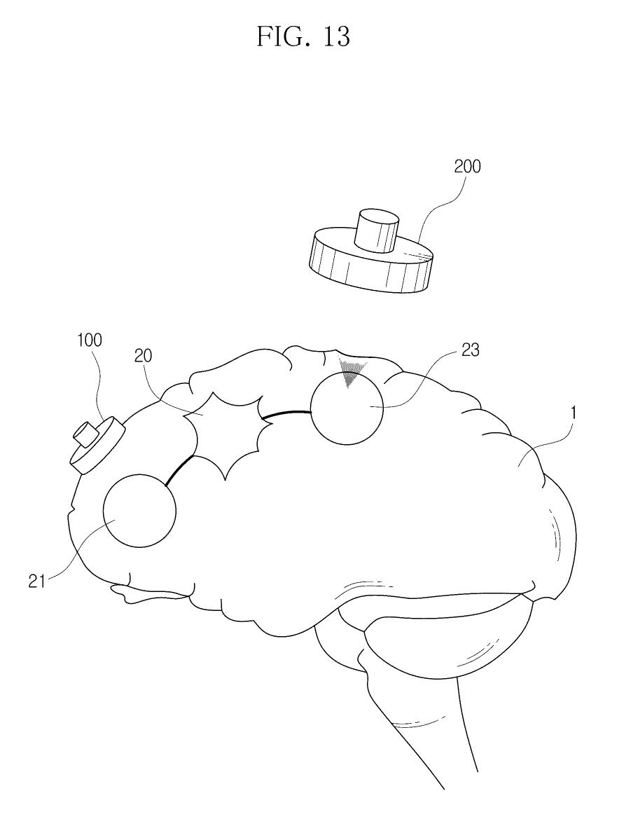

FIG. 13 a diagram illustrating still another example of application of the system 10 to the target brain 1 in which the affected part 20 of nervous necrosis is present.

According to this example of application, further to the previous example of application, when activity state information of a high-level function area involved in giving a command for a specific function is obtained, stimuli are directly applied to a low-level function area involved in executing the corresponding command in the brain neural circuit through which command signals generated from the corresponding high-level function area are transmitted, to help performing the function.

For example, the prefrontal association area 21 is a high-level function area from which a motor command to body is generated in the motor neural circuit, and the primary motor area 23 is a low-level function area at which the corresponding command is executed.

The computer 300 determines whether there is an intent to move the body by analyzing the brain activity state information of the prefrontal association area 21.

If it is in normal state, command signals generated from the prefrontal association area 21 are transmitted to the primary motor area 23 through the motor signal circuit, causing movements. However, the corresponding signal transmission is blocked by the affected part 20.

When an intent to make physical movements is recognized in the prefrontal association area 21, the computer 300 immediately stimulates the primary motor area 23 using the brain stimulation device 200, to cause the brain signals generated by the prefrontal association area 21 to indirectly act on the primary motor area 23.

When the primary motor area 23 is activated, physical movements take place, helping recovery or rehabilitation of the patient.

Through a brain map of the primary motor area 23 known as so-called “Homunculus”, stimuli can be selectively applied to an area of a part of the primary motor area 23 related to the body's organ that the prefrontal association area 21 intends to move.

Through analysis of brain activity information of the prefrontal association area 21, it can be seen that the target brain 1 generates a command to move, for example, a hand, and the brain stimulation device 200 selectively stimulates only an area related to hand motions in the primary motor area 23.

Along with this, if a peripheral nervous system from the primary motor area 23 to the hand is appropriately stimulated, an accurate hand motion will be made as intended by the subject.

The examples of application described with reference to FIGS. 12 and 13 do not need to take place independently, and the plurality of brain stimulation devices may simultaneously stimulate the primary motor area (low-level function area) 23 involved in executing a motor command in the motor neural circuit and a surrounding area of the affected part 20, thereby maximizing the rearrangement effect of the neural circuits.

According to the system 10 in accordance with this embodiment, stimuli are applied based on brain activity state of the subject, thereby reducing a sense of irritation or a sense of fatigue the subject feels, and preventing adverse effects caused by random stimuli from occurring.

Also, stimuli can be accurately applied to various target locations according to brain activity state, thereby accomplishing applications in various fields possible.

Further, stimuli reflecting the patient's intent can be applied, thereby maximizing the rehabilitation or treatment effect.

What is claimed is:

1. A brain to brain interface system comprising:
a brain activity detection device configured to detect activity state information of a brain;
a brain stimulation device configured to stimulate an area of at least a part of the brain to activate or inactivate brain cells of the corresponding area; and
a computer configured to control the brain activity detection device and the brain stimulation device,
wherein brain activity state information of a subject's brain (“a target brain”) is obtained through the brain activity detection device, and an area of at least a part of the target brain is stimulated through the brain stimulation device based on the brain activity state information of the target brain to regulate a function of the target brain.

2. The brain to brain interface system according to claim 1, wherein the computer identifies activity state of a brain neural circuit for performing a specific function through the brain activity state information, and allows the brain stimulation device to stimulate an abnormally activated area or an inactivated area in the brain neural circuit to inactivate or activate the corresponding area.

3. The brain to brain interface system according to claim 1, wherein the computer identifies activity state of a brain area for performing a specific function through the brain activity state information, and allows the brain stimulation device to stimulate other brain area for performing a different function from the specific function to activate or inactivate the other brain area.

4. The brain to brain interface system according to claim 1, wherein an affected part of nervous necrosis is present in the target brain, and
the brain stimulation device stimulates an arbitrary part of the target brain to remedy an unbalanced state of the entire target brain caused by the presence of the affected part.

5. The brain to brain interface system according to claim 4, wherein the brain stimulation device simultaneously stimulates an affected side in which the affected part is present and an unaffected side in which the affected part is absent, such that the affected side and the unaffected side are in opposite activity state.

6. The brain to brain interface system according to claim 1, wherein an affected part of nervous necrosis is present in the target brain, in which a signal transmission tract of a brain neural circuit for performing a specific function is blocked by the affected part, and
when activity state information of a high-level function area involved in giving a command for the specific function is obtained, the computer allows the brain stimulation device to stimulate an arbitrary of the target brain, to help form a new signal transmission tract to bypass the affected part.

7. The brain to brain interface system according to claim 6, wherein the brain stimulation device simultaneously stimulates a low-level function area involved in executing the command in the brain neural circuit, and a surrounding area of the affected part.

8. The brain to brain interface system according to claim 1, wherein an affected part of nervous necrosis is present in the target brain, in which a signal transmission tract of a brain neural circuit for performing a specific function is blocked by the affected part, and
when activity state information of a high-level function area involved in giving a command for the specific function is obtained, the computer allows the brain stimulation device to stimulate a low-level function area involved in executing the command in the brain neural circuit to help perform the function.

9. The brain to brain interface system according to claim 1, wherein the brain stimulation device is a low intensity focused ultrasound device which brings low intensity ultrasound beams into convergence to at least one focus.

10. The brain to brain interface system according to claim 8, wherein the low intensity focused ultrasound device moves a position of the focus three-dimensionally.
